(19) United States
(12) Patent Application Publication
(10) Pub. No.: US 2026/0162228 A1
(43) Pub. Date: Jun. 11, 2026

(54) SYSTEMS AND METHODS FOR ENHANCING IMAGING USING CONTRASTIVE LEARNING METHOD FOR PERCEPTUAL LOSS AND AN ORGAN-AWARE LOSS

(71) Applicant: Subtle Medical, Inc., Menlo Park, CA (US)

(72) Inventors: Xinrui Zhan, Shanghai (CN); Aimei Li, Jiangsu (CN); Jian He, Jiangsu (CN); Lei Xiang, Shanghai (CN)

(21) Appl. No.: 19/179,162

(22) Filed: Apr. 15, 2025

(30) Foreign Application Priority Data
Apr. 28, 2024 (WO) PCT/CN2024/090377

(51) Int. Cl. G06T 5/60; A61B 6/00

(52) U.S. Cl. CPC G06T 5/60; A61B 6/5258; G06T 2207/10081; G06T 2207/10104; G06T 2207/20081; G06T 2207/20084; G06T 2207/30004

(57) ABSTRACT

A method for training a deep learning model for enhancing medical imaging is provided. The method comprises: training a backbone model for a perceptual loss function utilizing a set of negative pairs and a set of positive pairs; generating a loss function comprising the perceptual loss function and an organ loss function; and optimizing parameters of a model using the loss function, wherein the model is trained to enhance a quality of medical images.

### CROSS-REFERENCE TO RELATED APPLICATION

This application claims priority to PCT International Application No. PCT/CN2024/090377 filed on Apr. 28, 2024, the content of which is incorporated herein in its entirety.

### BACKGROUND

Machine learning or deep learning has been employed in medical imaging to improve image quality. For instance, low-quality image or degraded image such as images acquired with reduced dose of contrast agent, accelerated acquisition, or acquired under standard conditions but degraded due to other reasons may be improved by applying a deep learning model to predict synthesized image with improved image quality. Loss functions are used for training deep learning models. However, conventional pixel-wise loss functions used in medical image enhancement and denoising often result in over-smoothing. While perceptual loss has shown effectiveness in natural image tasks, its performance degrades in medical imaging due to the incompatibility of nature image-trained backbone models and the challenge of assembling a medical dataset comparable to ImageNet.

### SUMMARY

A need exists for learning algorithm for training models that can be applied in medical imaging. The present disclosure provides improved imaging systems and methods that can address various drawbacks of conventional methods, including those recognized above. In some embodiments of the methods herein, a self-supervised-learning approach, Medical Volume framework for Contrastive Learning of visual Representation (MedCLR) is provided to train a backbone model. The backbone model is better suited for perceptual loss in medical contexts. The methods herein may apply the perceptual loss to enhance low quality medical images such as by taking as input low-dose Positron Emission Tomography (LPET) images and output PET images with improved quality. In some embodiments, the methods may employ an integration of loss functions such as an Organ Loss that focuses on enhancing Standard Uptake Value (SUV) quantification accuracy in key anatomical regions. The integration of the two loss functions such as the perceptual loss and organ loss functions significantly improves the realism and diagnostic value of the low-quality images (e.g., LPET images), marking a significant advancement in medical imaging artificial intelligence (AI).

As utilized herein, the term “perceptual loss” generally refers to a type of loss function that measures the difference between images based on their perceptual similarity, rather than pixel-by-pixel differences.

As utilized herein, “low quality” image herein may refer to degraded image which may comprise images acquired with reduced dose of contrast agent, accelerated acquisition, or acquired under standard conditions but degraded due to other reasons. Examples of low quality in medical imaging may include a variety of artifacts, such as noise (e.g., low signal noise ratio), blur (e.g., motion artifact), shading (e.g., blockage or interference with sensing), missing information (e.g., missing pixels or voxels in painting due to removal of information or masking), reconstruction (e.g., degradation in the measurement domain), and/or under-sampling artifacts (e.g., under-sampling due to compressed sensing, aliasing).

Though positron emission tomography (PET) and PET data examples are provided herein, it should be understood that the present approach can be used in any other imaging modalities. For instance, the presently described approach may be employed on data acquired by other types of tomographic scanners including, but not limited to, computed tomography (CT), single photon emission computed tomography (SPECT) scanners, functional magnetic resonance imaging (fMRI), or magnetic resonance imaging (MRI) scanners.

In some embodiments, a computer-implemented method for training a deep learning model for enhancing a quality of a medical image is provided. The method comprises: (a) training a backbone model for a perceptual loss function utilizing a self-supervised learning algorithm; (b) generating a loss function comprising the perceptual loss function in (a) and a second loss function; and (c) optimizing parameters of the deep learning model using the loss function, where the deep learning model is trained to enhance a quality of a medical image.

In some embodiments, the self-supervised learning algorithm comprises a contrastive learning. In some cases, the contrastive learning comprises generating a set of negative pairs and a set of positive pairs as training datasets for training the backbone model. In some instances, the set of positive pairs comprise a set of contiguous slices from a same sample. For example, the method further comprises assigning different weights to the set of positive pairs based at least in part on a similarity between the set of contiguous slices. As an example, the similarity between the set of contiguous slices is based at least in part on a separation of the slices.

In some cases, the set of negative pairs comprise a set of contiguous slices from different samples. In some instances, the method further comprises assigning different weights to the set of contiguous slices based at least in part on absolute difference of normalized slice indices.

In some embodiments, the medical image comprises a Positron Emission Tomography (PET) image or a computed tomography (CT) image acquired at a lower dose. In some embodiments, the deep learning model is trained to predict an output medical image having a quality that is higher than a quality of the medical image as an input to the deep learning model. In some embodiments, the second loss function is an organ-specific loss.

In another aspect, a computer-implemented method for training a model for perceptual loss is provided. The method comprises: generating a set of negative pairs and a set of positive pairs as training datasets, the set of positive pairs comprise a set of contiguous slices from a same sample, and the set of negative pairs comprise a set of contiguous slices from different samples; and training, using a contrastive learning algorithm, a backbone model for the perceptual loss based on the training datasets by tuning parameters of the backbone model, the perceptive loss is used to train a deep learning model for improving a quality of an image data.

In some embodiments, the set of positive pairs are generated by applying a transformation to a volume image of the same sample. In some embodiments, the contrastive learning algorithm comprise computing a similarity matrix for the set of positive pairs and the set of negative pairs. In some embodiments, the contrastive learning algorithm comprises assigning different weights to the set of positive pairs based at least in part on a similarity between the set of contiguous slices. In some cases, the similarity between the set of contiguous slices is based at least in part on a separation of the slices.

In some embodiments, the contrastive learning algorithm comprises assigning different weights to the set of contiguous slices based at least in part on absolute difference of normalized slice indices. In some embodiments, the image data comprises a Positron Emission Tomography (PET) image or a computed tomography (CT) image acquired at a lower dose. In some embodiments, the deep learning model is trained to predict an output image having a quality that is higher than a quality of the image data as an input to the deep learning model. In some embodiments, the backbone model is a convolutional neural network.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows examples of intrinsic similarities across slices within a medical volumetric data.

FIG. 2 shows an example of Medical Volume framework for Contrastive Learning of visual Representation (MedCLR) architecture.

FIG. 3 shows an example of a resultant organ mask.

FIG. 4 shows comparison of qualitative Results.

FIG. 5 shows an example of quantitative prowess of the loss function.

FIG. 6 shows an example of a method comprising the various features to improve the inference result.

FIG. 7 shows an example of an architecture of a denoising enhancement (DNE) model.

FIG. 8 shows an example of a framework for the super-resolution enhancement (SRE) module.

FIG. 9 shows examples of an output image of DNE and a final output image (DNE+SRE+Post processing) demonstrating the improved quality over the input image.

FIG. 10 shows an example of 3D High-Resolution Network architecture.

FIG. 11 shows an example of the patch training and sliding-patch inference method.

FIG. 12 shows an increase of sliding tile size may reduce the amounts of artifacts.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Deep learning has been employed to improve image quality. For instance, deep learning techniques have been utilized to improve medical imaging for better diagnosis. In some cases, low-quality image or degraded image such as images acquired with reduced dose of contrast agent, accelerated acquisition, or acquired under standard conditions but degraded due to other reasons may be improved by applying a deep learning model to predict synthesized image with improved image quality. For example, Positron Emission Tomography (PET) has demonstrated a clear clinical value in the management of cancer patients. Patients who undertake PET for treatment are injected with a large dose of radioactive tracer such as 18F-FDG or Gadolinium-Based Contrast Agents (GBCAs) into tissues or organs before scanning. This process generates radiation exposure, which may be harmful to patients, especially in patients who need multiple examinations or pediatric patients with a higher lifetime risk for developing cancer. Although lowering the dose of radioactive tracer (low-dose PET) can reduce radiation exposure, it also yields increased noise, lower SUV accuracy, artifacts, and a lack of imaging details. Such compromises can result in undetected lesions and inaccurate clinical diagnoses. A potential solution is to enhance LPET image quality.

Deep learning and convolutional networks (CNN) allow for leveraging CNN to improve LPET. These methods often rely on LPET/Full-dose PET (FPET) training pairs derived from list-mode scanning data to train the CNN. However, current training algorithm and training methods may have drawbacks. For example, LPET/FPET are not strictly pixel-wise aligned while the commonly employed loss functions are pixel-wise L1/L2 loss, leading the enhanced images to be overly smoothed and prone to image distortions. While perceptual loss has been employed for natural image tasks to improve image realism, its performance drops when applied to medical images since features learned from natural images don't always fit the medical image context. Training a perceptual backbone model is needed but it is hard to obtain a labeled dataset for medical images that is comparable to ImageNet. An additional limitation of the conventionally utilized L1/L2 loss is its indiscriminate application to all pixels within an image. This equal treatment can cause a decrease in SUV (standardized uptake value) accuracy for crucial organs which can result in diagnostic errors.

Though positron emission tomography (PET) imaging is primarily provided herein, it should be understood that the present approach, models, methods and systems may be used in other imaging modality contexts or various other image restoration tasks. For instance, the presently described approach may be employed on data acquired by other types of tomographic scanners including, but not limited to, computed tomography (CT), single photon emission computed tomography (SPECT) scanners, magnetic resonance (MR) scanner, functional magnetic resonance imaging (fMRI) scanners and the like. Methods, systems and/or components of the systems or models may be used in other imaging tasks (e.g., super-resolution, image denoising, accelerated imaging, lower contrast agent dosage, etc.).

The term “low quality” image as utilized herein may refer to degraded image which may comprise images acquired with reduced dose of contrast agent, accelerated acquisition, lower resolution, or acquired under standard conditions but degraded due to other reasons. Examples of low quality in medical imaging may include a variety of artifacts, such as noise (e.g., low signal noise ratio), blur (e.g., motion artifact), shading (e.g., blockage or interference with sensing), missing information (e.g., missing pixels or voxels in painting due to removal of information or masking), reconstruction (e.g., degradation in the measurement domain), under-sampling artifacts (e.g., under-sampling due to compressed sensing, aliasing), and/or other artifacts (e.g., image corruption).

The present disclosure addresses the above issues by providing an improved training method. The training method may comprise a self-supervised method based on contrastive learning. The self-supervised method for training the backbone model may not require labeled datasets. The training method herein may be used to train a model as a backbone model for perceptual loss. In some cases, the training method may comprise a Medical Volume framework for Contrastive Learning of visual Representation (MedCLR) for training a backbone model. In some embodiments, the training method may further comprise an organ loss to increase SUV quantification. For instance, the method may utilize Computed Tomography (CT) and anatomy segmentation mask gained from CT, and apply Organ Loss to impose extra constraints on key organs' areas and boundaries to increase the SUV quantification. The effect of the loss functions is verified by Standard of Care Plus (SOCP) dataset that doubled the routine scanning time.

A deep learning model trained utilizing the training algorithm herein may be capable of predicting high-quality images based on low-quality images with various artifacts or various noise distributions. Once the model is developed utilizing the training algorithm herein, it may be used for improving image quality. For example, the model may improve the image quality of an initial degraded image due to accelerated acquisition, reduced contrast agent dose, lower radiation dose, different radiopharmaceutical rejection, different scanning model/protocol and the like by generating a corresponding image with improved quality.

Current imaging enhancing methods may face image over-smoothing issues. Generative Adversarial Networks (GANs) with adversarial loss mechanisms have been employed to overcome image over-smoothing issue. GANs may reduce over-smoothing by using a discriminator network to encourage generated images to look more like the real images. Other methods may include utilizing the contextual loss for the over-smoothing where images are not strictly aligned pixel-by-pixel. However, both GANs and contextual loss techniques bear the risk of instability and may introduce distortions into the images. To address the challenge of adapting perceptual loss models from natural images to medical imaging, researchers have sought alternative training strategies due to the difficulty in collecting a labeled medical image dataset for supervised training. For instance, self-supervised learning strategy has been employed to train an autoencoder, with the objective for the encoder to learn to compress features such that they can be reconstructed back to the original image. This autoencoder is then employed within a perceptual loss framework for CT denoising. Although this approach shows potential, the simplicity of the reconstruction task may not enable the model to learn sufficiently complex features. Other methods try to solve the problem using a segmentation model to provide perceptual loss; however, such methods require additional labeling for segmentation, adding to the complexity of the process.

The training methods herein may comprise a Framework for Contrastive Learning of Visual Representations. Conventional framework may enhance agreement among different augmented views of the same image, known as positive pairs, while reducing agreement with views of distinct images, or negative pairs. The objective is to maximize the agreement between positive pairs (instances from the same sample) and minimize the agreement between negative pairs (instances from different samples). Contrastive learning leverages the assumption that similar instances should be closer together in a learned embedding space, while dissimilar instances should be farther apart. By framing learning as a discrimination task, contrastive learning allows models to capture relevant features and similarities in the data. However, such conventional framework is largely dependent on how these pairs are constructed mainly through using a variety of image augmentations. Yet, many conventional data augmentations as color distortion are not suitable for medical imaging. Unlike the traditional data augmentation, the present disclosure provides an improved framework to form positive pairs and negative pairs, leveraging the intrinsic similarities within medical volumetric data.

In an aspect of the present disclosure, a method for training a deep learning model for enhancing medical imaging is provided. The method comprise: training a backbone model for a perceptual loss function utilizing a set of negative pairs and a set of positive pairs; generating a loss function comprising the perceptual loss function and an organ loss function; and optimizing parameters of a model using the loss function, wherein the model is trained to enhance a quality of medical images.

### Training Method

The present disclosure provides methods to enhance medical imaging (e.g., low-quality image or LPET images) by integrating a perceptual loss and an organ-specific loss. In some embodiments, the perceptual loss is trained with MedCLR which is better applicable to medical imaging. A model for enhancing image quality is trained utilizing the loss function here may have improved performance demonstrated by metrics such as Peak Signal-to-Noise Ratio (PSNR) and Deep Image Structure and Texture Similarity (DISTS). Such metrics show that the presented training algorithm can beneficially enhance visual realism in low-dose PET (LPET) images or other low-quality images.

The training methods herein may comprise a Framework for Contrastive Learning of Visual Representations. In some embodiments of the methods herein, a self-supervised-learning approach, Medical Volume framework for Contrastive Learning of visual Representation (MedCLR) is provided to train a backbone model. A backbone model may be a convolutional neural network (CNN) that is used to extract features from the input images, and these features are then used to compute the perceptual loss. The improved training methods may comprise forming positive and negative pairs, leveraging the intrinsic similarities within medical volumetric data.

### Intrinsic Similarities

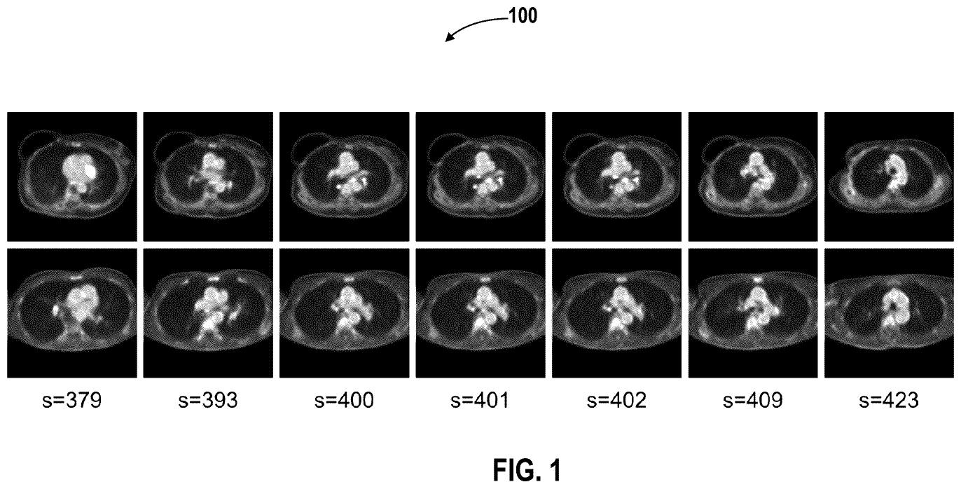

Medical volume images, such as PET and CT scans, are composed of 3D volumes. The 3D volumes may comprise a stack of cross-sectional slices. Due to the continuity of the imaged object such as human body, slices that are close to each other or within a continuous region may present similar visual or appearance features. Furthermore, while scans from different individuals exhibit numerous differences, the dissimilarity may decrease as the related physical locations in the body of the slices become closer. FIG. 1 shows examples 100 of intrinsic similarities across slices within a medical volumetric data. Rows display slices selected from the same medical volume, with the slice index s indicated below. Within a row, slice s=400 shows greater similarity to slice s=401 than to slice s=379. Across rows, slices at s=401 exhibit fewer differences compared to a pair composed of slices s=423 and s=401.

The methods herein may comprise forming training data for contrastive learning. The training data may comprise positive pairs and negative pairs generated based on the intrinsic similarities. As described above, the term “positive pairs” may refer to different augmented views of the same image. The term “negative pairs” may refer to the views of distinct images.

In some cases, the similarities may be defined as within the same 3D volume, the similarity between slices is inversely proportional to the distance between their indices' indexes. When the difference between the indexes is 0, two slices are identical. When comparing slices from two distinct volumes, their dissimilarity increases as the difference in their related indices in the body grows.

### Self-Supervised Learning for Training Backbone Model

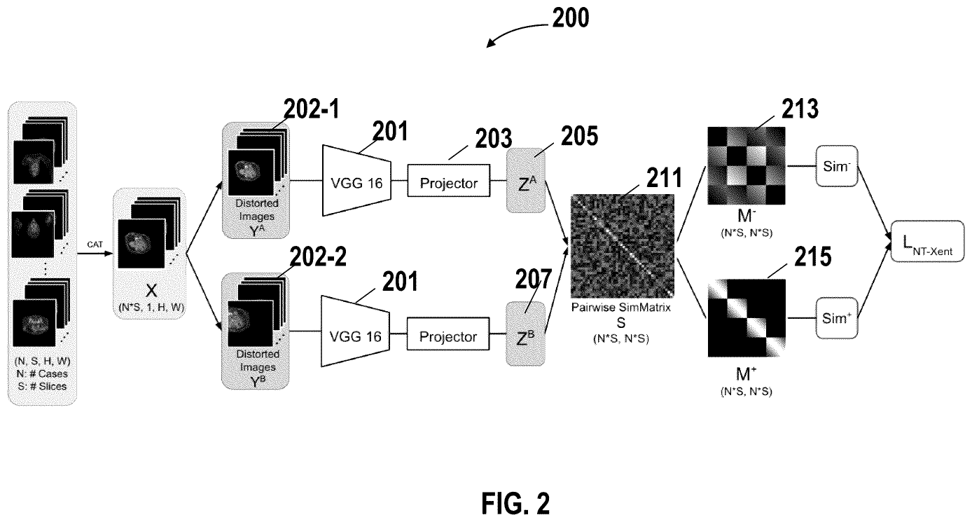

In some embodiments, systems and methods herein may provide a Medical Volume framework for Contrastive Learning of visual Representation (MedCLR). FIG. 2 shows an example of MedCLR architecture 200. This architecture along with the unique training dataset may be used to train a backbone model for perceptual loss. As shown in the example, the backbone model may be a convolutional neural network (CNN) such as VGG16 feature layers 201. The backbone model may be pre-trained utilizing the MedCLR architecture 200 herein. Once trained, the backbone model may be utilized to train any other model for enhancing a medical image.

In some embodiments, the MedCLR may comprise selecting distinct samples, each comprising a set of consecutive slices, which are then processed to form a combined image batch. The combined image batch may be processed with transformation and embedding, culminating in a similarity matrix S that reflect the intricate relationships between the set of consecutive slices and between the distinct samples.

As shown in FIG. 2, N distinct samples are selected, each comprising S contiguous slices, to form a batch of 2.5D volumes (N, S, H, W). The term 2.5D volumes may refer to a stack of contiguous or consecutive 2D slices. The 2D slices may be acquired at different depths or along an imaging direction then stacked to form a volume. The consecutive 2D slices may be indexed such that different indices correspond to a 2D image slice acquired at different locations (e.g., different depths, different locations along the same direction, etc.). The N distinct samples are combined into a 2D image batch X of shape (N*S, 1, H, W). Through a transformation t, batch X is altered into two variant batches γ^{a} 202-1 and γ^{b} 202-2, which are then embedded (e.g., by the embedding function implemented by VGG 16 as the backbone model 201), pooled, and projected (e.g., by the projector 203) to produce hidden representations Z^{A} 205 and Z^{B 207}. The transformation t applied to the corresponding two inputs may or may not be the same. For instance, the transformation applied to the two inputs may have different parameters for the resize scale, gaussian blur filter, sharpness adjustment or cropping. As an example, a first transformation may include random resize and crop with bicubic interpolation, the resize scale may be set, for instance, to between (0.9, 1.), the crop is set to be 256, the random gaussian blur (e.g., with kernel=3 and probability=0.2) may be applied and random sharpness adjustment may be set with a number different from a second transformation applied to the second input.

A similarity matrix S 211 of shape (N*S, N*S) is computed using the dot product of L2 normalized hidden dimensions, encapsulating the pair-wise similarity within and between samples.

In some embodiments, based on the aforementioned similarity definition, positive pairs may comprise slices drawn from the same volume after different transformation. In some cases, the positive pairs may be weighted accounting for the similarity decrease with respect to increasing slice separation (e.g., greater indexes difference corresponds to less similarity). In some cases, given that the consecutive order of slices are from the same sample or the same S contiguous slices, all the slices are positive paired with each other with a designed weight accounting for decreasing similarity with increasing slice separation. The method may comprise calculating a weight for each slice from the same consecutive set of slices or within the same volume. In some cases, the weight may be calculated using an exponential of the negative half-power of the normalized index difference.

The method may also assign weights to negative pairs which comprise slices from different volumes. In some cases, the weights assigned to negative pairs may be based on the absolute difference of normalized slice indices. For computational efficiency and GPU optimization, both positive and negative weights are structured as matrices of shape (N*S, N*S) represented by positive weight matrix M⁺ 213, and negative weight matrix M⁻ 215. Their computation is detailed in Eq. (2) and Eq. (1), where Sli denotes the normalized slice index and the

$$[ \frac{i}{S} ]$$

indicates the associated case of slice. As shown in Eq. 3, the Normalized Temperature-scaled Cross-entropy (NT-Xent) loss are calculated based on pairwise similarity matrix S, positive weight matrix M⁺, and negative weight matrix M⁻.

$${M}_{ij}^{+} = \{ \begin{matrix}{e}^{- \frac{1}{2} {( \frac{i - j}{σ} )}^{2} ,} & if ⌊ \frac{i}{S} ⌋ = ⌊ \frac{j}{S} ⌋ \\ 0 , & Otherwise\end{matrix} \qquad ( 1 )$$

$${M}_{i j}^{-} = \{ \begin{matrix}\vert {Sl}_{i} - {Sl}_{j} \vert , & if not ⌊ \frac{i}{S} ⌋ = ⌊ \frac{j}{S} ⌋ \\ 0 , & Otherwise\end{matrix} \qquad ( 2 )$$

$$\begin{matrix}{loss}_{i , j} = - log \frac{{\sum }_{i j} exp ( {S}_{i j} ) * {M}_{i j}^{+} / τ}{{\sum }_{i j} exp ( {S}_{i j} ) * {M}_{i j}^{-} / τ} \\ = - log \frac{exp ( S ) ∘ {M}^{+} / τ}{exp ( S ) ∘ {M}^{-} / τ}\end{matrix} \qquad ( 3 )$$

### Perceptual Loss

The methods herein may provide an improved perceptual loss or an improved backbone model that is used for perceptual loss. The backbone model is trained utilizing the Contrastive Learning of visual Representation (MedCLR) as described above. For instance, the backbone model is trained utilizing training dataset comprising the negative pairs and positive pairs with the weights assigned. The unique training set may provide a model that is better suited for medical imaging compared to model pretrained with natural images (e.g., ImageNet, ResNet, etc.).

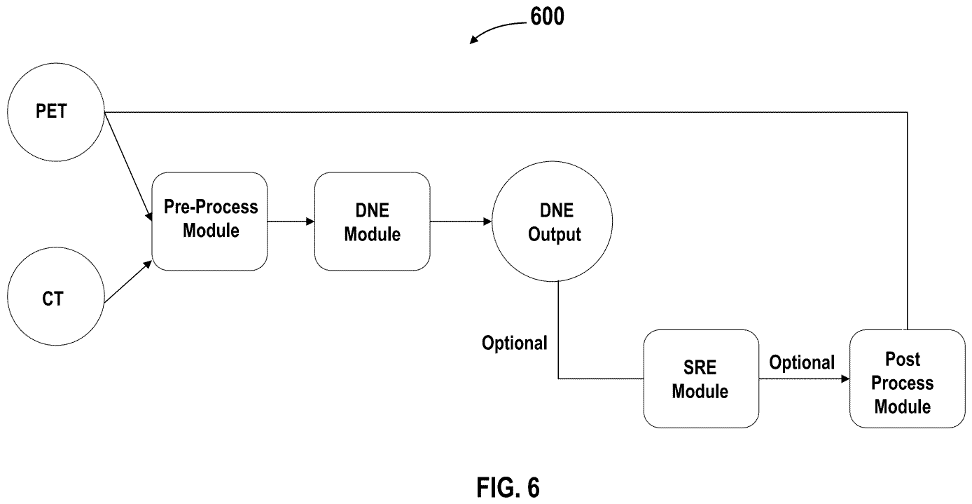

As illustrated in FIG. 2, in some embodiments, the method herein may employ a convolutional neural network (CNN) such as the VGG16 feature layers as the embedding function to derive features from both the predicted image i.e. enhanced low-dose image (e.g., Higher-quality PET (HPET)) and its corresponding ground truth. The ground truth data may comprise standard PET (e.g., full dose PET) or PET with quality improved over LPET. In some cases, the ground truth image may be acquired with standard of care protocol (e.g., full dose or standard dose of contrast agent and standard acquisition time). In alternative cases, the ground truth image may be acquired with a higher quality of standard of care. This beneficially improves the performance of the trained model. For instance, the acquisition time for the higher quality of standard of care may be longer than the acquisition time of a standard protocol. Details about higher quality of image as ground truth and other features to improve the model performance are described later herein and with respect to FIG. 6.

Subsequently, an L1 distance is computed. Given an arbitrary enhancement model M (e.g., a denoising enhancement (DNE) module or a super-resolution enhancement (SRE) module) with parameters θ and a pretrained VGG feature extraction modules Ø, the perceptual loss of image Y^{gt} and Y^{pred} is articulated in Eq. (4).

$${ℒ}_{perceptual} = \frac{1}{WH} {\sum }_{x = 1}^{W} {\sum }_{y = 1}^{H} \vert {∅ ( {Y}^{gt} )}_{x , y} - {∅ ( {M}_{θ} ( {Y}^{lpet} ) )}_{x , y} \vert \qquad ( 4 )$$

It should be noted that the backbone model can be any different CNNs (e.g., VGG16, VGG19, ResNet50, ResNet101, and InceptionV3) depending on the specific application (e.g., super resolution, transformation) or the requirement of number of parameters. A backbone model trained utilizing the MedCLR along with the negative and positive pairs as described above is improved over conventional pretrained backbone models when it is implemented as perceptual loss in medical imaging enhancement.

### Organ Emphasize Loss

The training method of the present disclosure may provide a loss function integrating the perceptual loss and organ loss (organ emphasize loss). The terms organ emphasize loss, organ-specific loss, and organ loss are utilized interchangeably throughout the specification and generally refer to a region-specific loss that is to assign different weights to different regions of the image, penalizing errors in organ regions more heavily. The perceptual loss may be provided by the same backbone network model as described above. An organ loss may be employed to focus on enhancing Standard Uptake Value (SUV) quantification accuracy in key anatomical regions. The integration of the two loss functions (i.e., the perceptual loss and organ loss functions) significantly improves the realism and diagnostic value of the low-quality images (e.g., LPET images), marking a significant advancement in medical imaging artificial intelligence (AI).

In some cases, the loss function may comprise the perceptive loss, organ loss and other metrics to measure the visual differences (e.g., Mean Absolute Error (MAE), structural similarity (SSIM)). Following is an example of a loss function integrating Mean Absolute Error (MAE), structural similarity (SSIM), a perceptual loss, and organ loss. The contributions of these components are controlled by hyper-parameters (e.g., α=1.0, β=0.1, and ‘y=0.5), as defined in Eq. (8).

$${ℒ}_{proposed +} {ℒ}_{1} + {αℒ}_{ssim} + {βℒ}_{perceptual} + {γ}_{ℒ organ} \qquad ( 8 )$$

The above loss function may be utilized to optimize parameters of a deep learning model for enhancing medical imaging. For instance, parameters of the deep learning model may be tuned to minimize the measurements or the loss.

While both L1 and SSIM treat all pixels equally, it is beneficial to assign higher weight on target organ area compared to blank background or flat regions. In some embodiments, the organ loss may be provided by an anatomical segmentation mask. The anatomical segmentation mask is pre-generated utilizing any suitable segmentation methods (e.g., TotalSegmentator).

The anatomical segmentation mask may be generated based on an image of a modality different from the image that is to be enhanced by the trained model. In some cases, some organ classes may not be distinctly visible in PET images, such as the pulmonary vein, and others may represent different sections of a singular region, such as vertebrae C1, the method may consolidate the organ classes such as by consolidating an initial 117 classes into 18 distinct classes.

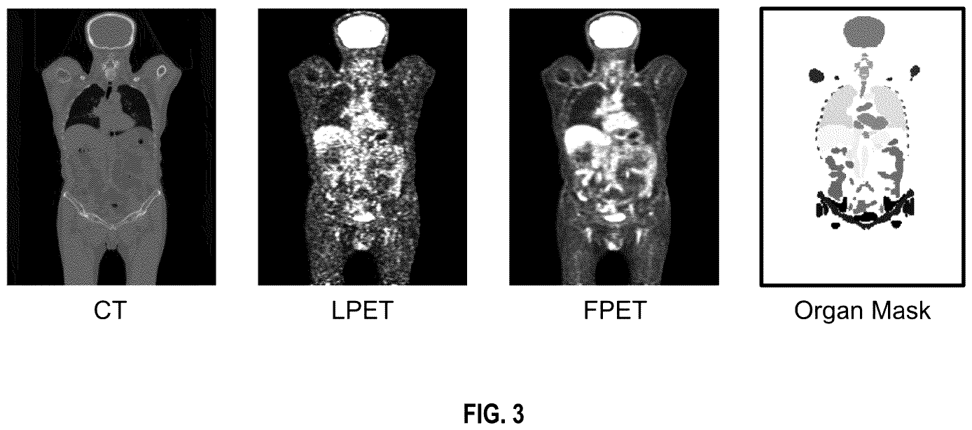

FIG. 3 shows an example of a resultant organ mask. The mask (e.g., organ mask, or any other suitable region mask) may be used to assign weights to region of interest. For instance, higher weight on target organ area may be assigned compared to blank background or flat regions. Similarly, specific constraint on different regions identified by the segmentation mask may be applied. Besides the class index 0 which represent the background, extra constraints on each distinct organ areas may be added. For instance, extra constraints on each distinct organ areas from index 1 to C are added: an L1 loss in order to improve the SUV quantification and an edge loss in order to improve the boundary clarity.

$${ℒ}_{Organ} ( {Y}^{pred} , {Y}^{gt} ) = {\sum }_{i = 1}^{C} ( {ℒ}_{edge} ( {Y}_{i}^{pred} , {Y}_{i}^{gt} ) + {ℒ}_{mse} ( {Y}_{i}^{pred} , {Y}_{i}^{gt} ) ) \qquad ( 5 )$$

where

$$( {Y}_{i}^{pred} , {Y}_{i}^{gt} )$$

represents the masked organ region of predicted image and ground truth corresponding to organ i. The [symbol] _{edge} is defined as:

$${ℒ}_{edge} = {\sum }_{i , j} \vert {∇}_{S} {Y}_{i , j}^{Pred} - {∇}_{S} {Y}_{i , j}^{gt} \vert \qquad ( 6 )$$

where ∀s represents the Sobel operator, defined as:

$${∇}_{s} Y = \qquad ( 7 )$$

with Gₓ and G_{y} being the horizontal and vertical Sobel kernels respectively.

The present disclosure provides an improved loss function comprising the perceptive loss, organ loss to train a model to enhance image quality. The integration of the two loss functions (i.e., the perceptual loss and organ loss functions) significantly improves the realism and diagnostic value of the low-quality images (e.g., LPET images). A deep learning model trained utilizing the methods herein may take as input a low-quality image and predict synthesized image with improved image quality.

### EXPERIMENTS AND RESULTS

The collected SOCP dataset contains 41 F18-FDG scans from the Philips Vereos PET/CT machine with 30 cases used for training and 11 for validation. SOCP data was collected by doubled the scan time to 2 minutes. For LPET enhancement, a 2.5D U-Net++ architecture is employed with 14 channels. Each input channel combined 7 normalized LPET slices with their corresponding CT images, where LPET was normalized using the global mean of the volumes. The loss function herein integrates MAE, SSIM, a perceptual loss, and Organ loss as described above. The contributions of these components are controlled by hyper-parameters α=1.0, β=0.1, and ‘y=0.5, as defined in Eq. (8).

For MedCLR training, the 2022 Au-toPET challenge and the 2022 Ultra-Low-Dose challenge datasets are used. The experiment conducted 3000 epochs at a learning rate of 2e-5, with a cosine annealing scheduler. Applied distortions included resizing, cropping, Gaussian blur, and flips (i.e., transformation). The case count N is fixed to 4 and slice number F is fixed to 11, with a σ value of 3.16 for positive weight computation.

The model's performance was assessed using a suite of metrics tailored to capture both pixel-wise discrepancies and feature-wise similarities. The Peak Signal-to-Noise Ratio (PSNR) and the Structural Similarity Index Measure (SSIM) serve as the conventional benchmarks for pixel fidelity and structural congruence, respectively. Deep Image Structure and Texture Similarity (DISTS) is further utilized for a nuanced understanding of textural alignment. SUV quantification was meticulously scrutinized through the SUV max value error rate (SUVmaxE) calculated as the average error rate across anatomical regions.

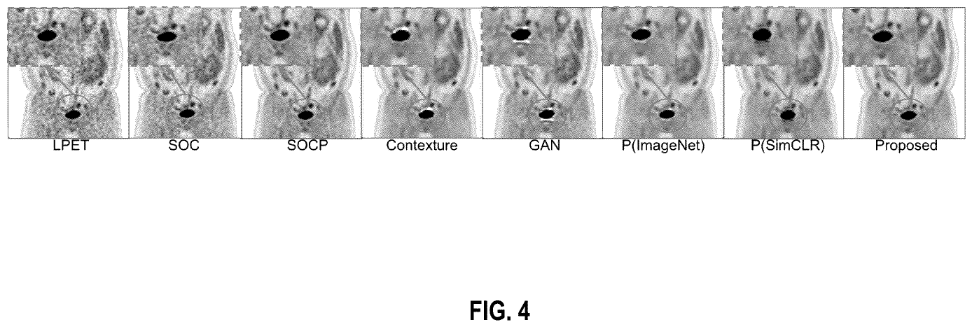

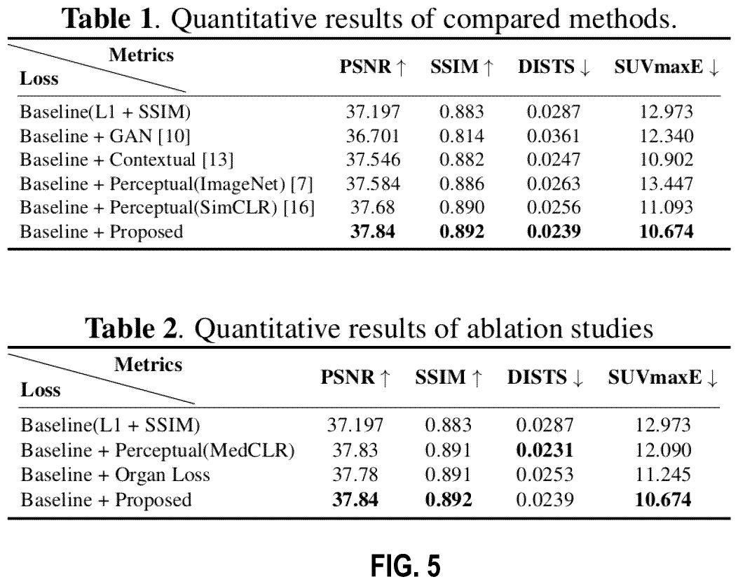

The quantitative prowess of the loss function as provided herein is shown in FIG. 4 and FIG. 5. FIG. 4 shows comparison of qualitative Results. Displayed is a sagittal plane view from a single case. Various add-on loss functions are compared: Contexture Loss, GAN, ImageNet Pretrained Perceptual Loss, ImageNet Pretrained using SimCLR, and the method described herein. In FIG. 5, Table 1 shows superiority across almost all metrics, with a negligible deficit in SUVmeanE when juxtaposed with Perceptual Loss trained using SimCLR. Ablation studies, presented in Table 2, further delineate the strengths of the perceptual loss trained via MedCLR, particularly in enhancing PSNR and DISTS, showing it can elevate visual realism. The organ loss, on the other hand, significantly advances SUV quantification accuracy, aligning with our targeted goals. The amalgamation of both losses heralds improvements in SSIM with modest increase in SUVmaxE and PSNR compared to standalone losses.

Qualitatively, the proposed training method distinguishes itself through the maintenance of structural integrity in critical regions such as the bladder, where competing methods falter, manifesting distortions. This fidelity is illustrated in FIG. 3, where the methods herein not only preserve organ boundaries but do so with enhanced clarity and less blurring, upholding the denoising efficacy without sacrificing detail—a testament to the robustness of the method against common artifacts.

### Further Improvement of Model Performance

In some cases, the input images may be acquired by positron emission tomography with 2-deoxy-2-[fluorine-18]fluoro-D-glucose integrated with computed tomography (¹⁸F-FDG PET/CT) for the detection of various cancers. The combined acquisition of PET and computed tomography (CT) has synergistic advantages over PET or CT alone. The present disclosure provides various methods and features to further improve performance of a deep learning model for enhancing the imaging quality. FIG. 6 shows an example of the method 600 comprising the various features. The input images may comprise the original PET data and original CT data. The input PET and CT data may be acquired in the same imaging session or ¹⁸F-FDG PET/CT acquisition.

In some cases, the ground truth data for training the model may be acquired with a higher quality of standard of care. A higher standard or high-quality data as ground truth may further improve the performance. For instance, the acquisition time for the higher quality of standard of care may be longer than the acquisition time of a standard protocol. As an example, the training data may comprise low-dose PET (LPET) that is acquired at shortened acquisition time such as 10s or 30s (or any number below 60s), a full-dose PET acquired at standard acquisition time 60s (e.g., standard of care 60s (SOC)), and/or a higher quality image acquired at longer acquisition time such as 120 s (e.g., standard of care plus 120s (SOCP)).

Referring back to FIG. 6, the architecture may comprise a pre-process module. The pre-process module may process both the CT and PET data (e.g., LPET and high-dose PET (HPET)) such as by normalization (e.g., normalized by the global 3D mean). The pre-processing methods can be the same as those described in PCT/CN2023/113700 filed on Aug. 18, 2023, the content of which is incorporated herein in its entirety. For instance, the original raw image data such as PET standardized uptake value (SUV) and CT volumes may be processed to be arranged into multiple channels or intensity ranges. For example, the PET SUV image may be mapped from (0, 30) SUV to (0, 1) range. This range may capture most of the PET intensities. The CT image (CT image that matched the PET) may be mapped from (−150, 300) to (0, 1). This range may capture the important patterns of the CT. In some cases, the CT image may be mapped to a CT soft range such as range (−100, 100) to focus on the soft-tissue intensities. In some cases, the intensity range may be dependent on the tissue or subject being imaged. For example, the CT image may be mapped to a CT Lung range such as range of (−1000, −200) to capture the intensities of the lung tissues.

In some cases, the pre-processing may comprise normalizing the CT image by min-max using −1000 as min and 3000 as max and dividing it by global 3D mean to be consistent with the PET data. The CT data may be resampled to the same dimension as the PET along the z-axis. The pre-processing module may comprise a pad-crop unit that is automatically applied to the PET data so the shape of the PET is divisible by a predetermined number (e.g., divisible by 36). Similarly, the CT data may not be reshaped into the same dimension as the PET, instead it may be processed by the pad-crop unit and automatically padded or cropped into a shape divisible by the same predetermined number (e.g., divisible by 36). In some cases, the pre-processing module may have a predetermined max dimension. For instance, the highest dimension allowed for CT may be predetermined (e.g., highest dimension is 512), and if the shape is greater than the predetermined number (e.g., 512), a bilinear resampling may be applied to downsample to the predetermined number (e.g., 512).

The architecture may comprise a deep learning model trained utilizing the training algorithm as described above to enhance an image quality of the input image. For example, the pre-processed PET and CT image may be supplied to a denoising enhancement (DNE) module and/or a super-resolution enhancement (SRE) module.

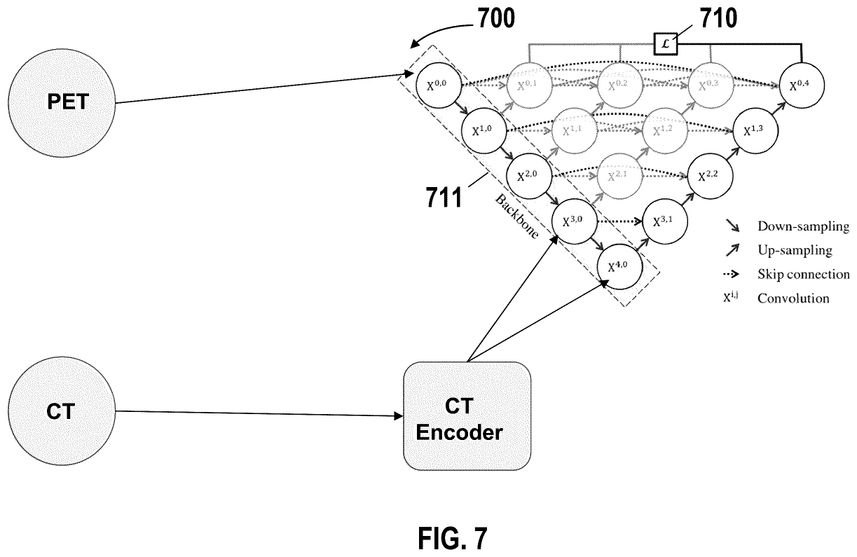

In some cases, the DNE and/or the SRE model may be trained utilizing the higher standard image as ground truth as described above. The loss function for training the DNE or the SRE model may comprise the perceptual loss and the organ loss function as described above. FIG. 7 shows an example of an architecture of a DNE model. In some cases, the PET and CT may each have a distinct encoder. The encoded CT's input features may be pooled to match the PET's input features' shape. For instance, in the bottom two layer, the pooled CT's features may be directly added on PET's features. The DNE model may have any suitable architecture such as UNet or UNet++ (e.g., consists of U-Nets of varying depths whose decoders are densely connected at the same resolution via the redesigned skip connections).

As described above, the DNE model may be trained by the presented training method herein to reduce noise in the input image. For example, the loss function 710 may comprise a perceptual loss with an improved backbone model 711. The backbone model may be trained using MedCLR as described above. The loss function 710 may further comprise an organ loss function. The organ loss may be generated based on an anatomical region's mask from the CT data that is paired with the PET. As descried elsewhere herein, the organ loss adds specific constraint on different regions. For instance, for bones, edge loss is added to increase the boundary clarity and for lung, liver, bladder, and other soft tissue/organ, an additional weight for L1-loss may be added. The loss function 710 may further comprise accuracy metrics such as L1 and SSIM. L1 is a pixel-wised loss that directly adds constraints to the distance of each single pixel between output and the target. The inclusion of SSIM may strike a balance between preserving high-level structural information and minimizing pixel-level differences. This combination encourages the model to generate images that not only match in terms of pixel values but also in perceptual quality.

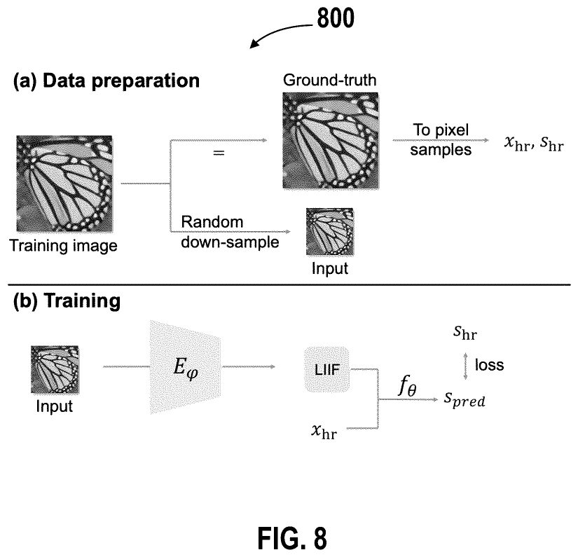

In optional embodiments, the image may be further enhanced by being processed by an SRE model (e.g., to increase resolution). As shown in FIG. 6, the output of the DNE module such as the denoised output PET image may be further supplied to a SRE module to increase the resolution. FIG. 8 shows an example of a framework for the SRE module. In some embodiments, the SRE may comprise a LIIF architecture (Learning Continuous Image Representation with Local Implicit Image Function. The SRE model may be trained using a unique training method. For example, instead of using patch training and edsr or rrdb, the whole image and a Unet as encoder may be employed. The training image may be randomly down-sampled to generate the input image. In some cases, the scaling size is not uniform sampling between [min, max] (e.g., maximum scaling size is 3 and minimum scaling size is 1). The scaling size may be determined based on the probability of scaling SRE task in real scenario (e.g., a higher probability to select scale size as 1 if in real scenario the SRE is no-scaling SRE task).

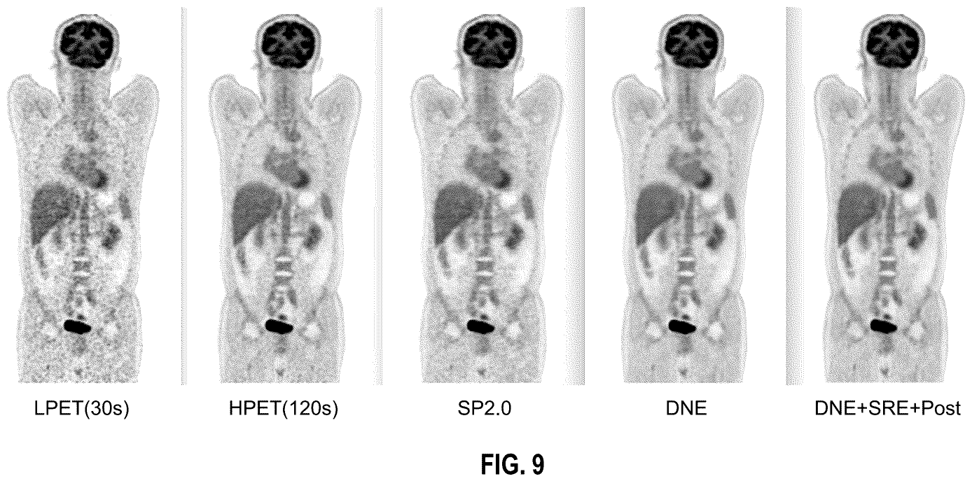

Referring back to FIG. 6, the architecture may optionally comprise a post-processing module. In some cases, the post-processing may comprise applying Gaussian filter and image deduction to the output of the DNE or DNE and SRE to obtain the high pass regions. Next, the post-processing may further comprise applying a threshold on the high pass regions to generate a mask. In some cases, the method may replace the mask with the input voxel's value and time with a constant (e.g., fixed constant) to generate an output. In some cases, a gaussian filter (e.g., a small kernel Gaussian) may be applied to further smooth the change in the mask. Next, the final output image may be generated by combining the thresholded highpass, highpass, and/or lowpass regions together. FIG. 9 shows examples of an output image of DNE and a final output image (DNE+SRE+Post processing) demonstrating the improved quality over the input image.

The training method herein may also be utilized to train a model for enhancing an ultra-low-dose input image. For instance, the deep learning model may be trained to enhance and reconstruct full-dose PET image taking an ultra-low-dose data as input. In some cases, ultra-low-dose may refer to low dose data that only have no more than 1/20, 1/50, or 1/100 of standard dosage usage or no greater than 1/20, 1/50, or 1/100 of standard scanning-time. In some embodiments, the interference result may be further improved by employing 3D High-Resolution Network (HRnet3D) model as the enhancement model, utilizing a combination of L2 and SSIM loss and/or reducing sliding patch artifacts (by using FLIP_Rotation inference).

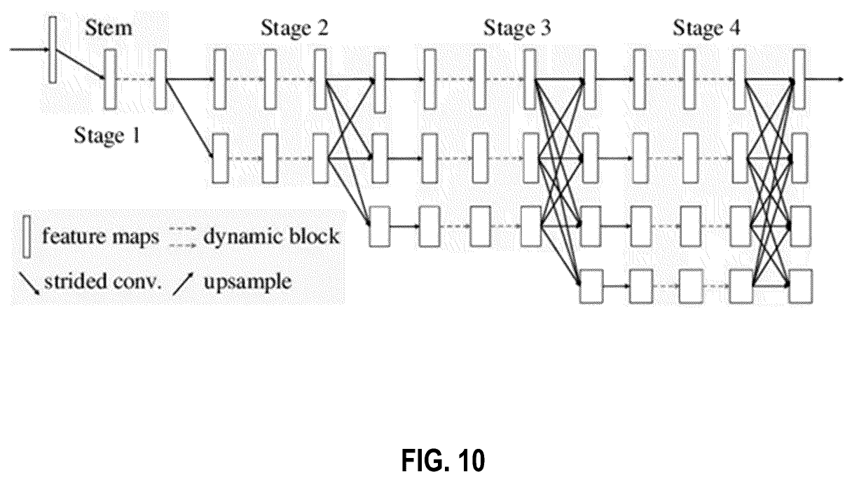

FIG. 10 shows an example of 3D High-Resolution Network architecture. The HRNet3D beneficially maintains high-resolution representations throughout the process, starting from a high-resolution stream and gradually adding high-to-low resolution streams connected in parallel. The model herein may be a 3D version of the HRNet which allows the model to receive additional information from the z-axis space and interpret the information to reconstruct the LPET

The inference performance may be further improved by employing L2 loss instead of L1 loss to minimize the noise while preserving the overall structure and smoothness of the image. L2 regularization penalizes large errors less harshly than L1, leading to smoother transitions and a more continuous output. Additionally, the loss function may further include SSIM loss to maintain the output's overall structure consistency and integrity.

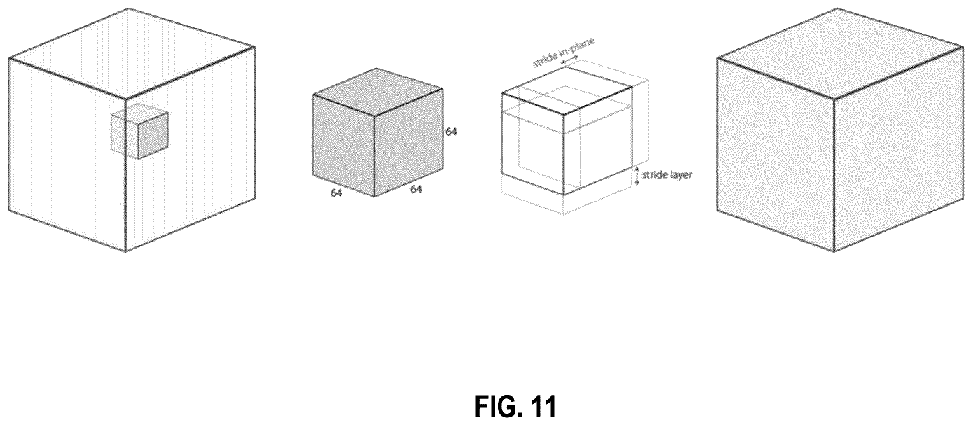

In some embodiments, the framework may further comprise 3D Patch Training and sliding-Patch Inference to reduce GPU memory requirement. Due to the GPU memory limitation, the model may not be trained and inference on 3D whole image. The method herein may process a patch (e.g., patch with a predetermined size such as (64, 64, 64)). FIG. 11 shows an example of the patch training and sliding-patch inference method. During inference, the sliding patch method may comprise processing the input image in sliding 3D patches and making inferences for each sliding patch/tile and output the whole 3D image.

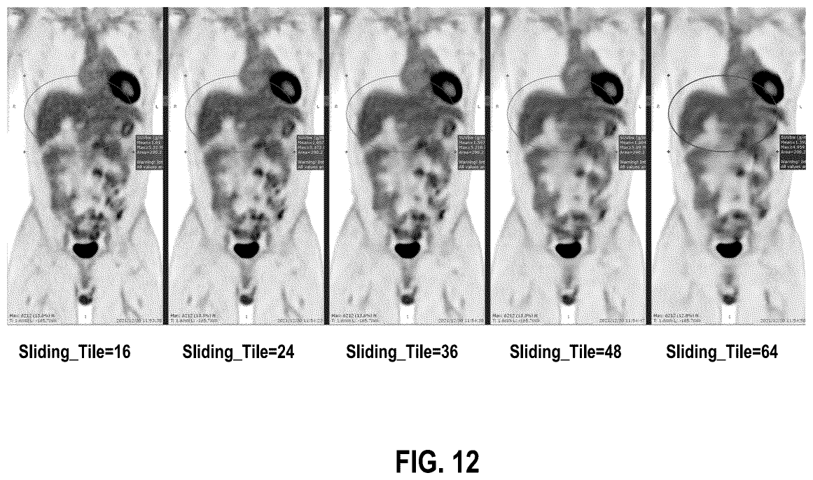

In some cases, line-artifacts may exist around the edge between each patch as the model may have minor disagreement on the edge of the adjacent patches and result in the artifacts. As illustrated in FIG. 12, an increase of sliding tile size may reduce the amounts of artifacts. However, increasing the tiling of the sliding patch while reduces the artifacts, it can increase the running time. For example, double the overlap ratio will result of 2\hat{ }3× patches to infer.

The present method may advantageously address the above issue by flipping or rotating the input 3D volume to create augmented version of 3D volume, applying the inference to both of the augmented version and the original version, and averaging the normal inference output and transformed-back version of augmented input. This beneficially allows for a reduction of artifacts without significantly increasing inference time (e.g., only 2× inference time).

The systems and methods can be implemented on existing imaging systems or various other imaging modalities without a need of a change of hardware infrastructure. Alternatively, the systems and methods can be implemented by any computing systems that may not be coupled to any imaging system. For instance, methods and systems herein may be implemented in a remote system, one or more computer servers, which can enable distributed computing, such as cloud computing.

The methods herein can be implemented using a computer system. The computer system can comprise a laptop computer, a desktop computer, a central server, distributed computing system, etc. The processor may be a hardware processor such as a central processing unit (CPU), a graphic processing unit (GPU), a general-purpose processing unit, which can be a single core or multi core processor, a plurality of processors for parallel processing, in the form of fine-grained spatial architectures such as a field programmable gate array (FPGA), an application-specific integrated circuit (ASIC), and/or one or more Advanced RISC Machine (ARM) processors. The processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

Systems and methods of the present disclosure may provide a noise generator that can be implemented in software, hardware, firmware, embedded hardware, standalone hardware, application specific-hardware, or any combination of these. The noise generator can be a standalone system that is separate from the imaging system or other software modules (e.g., denoising model or image enhancement software).

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein A and/or B encompasses one or more of A or B, and combinations thereof such as A and B. It will be understood that although the terms “first,” “second,” “third” etc. are used herein to describe various elements, components, regions and/or sections, these elements, components, regions and/or sections should not be limited by these terms. These terms are merely used to distinguish one element, component, region or section from another element, component, region or section. Thus, a first element, component, region or section discussed herein could be termed a second element, component, region or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A computer-implemented method for training a deep learning model for enhancing a quality of a medical image, the method comprising:
(a) training a backbone model for a perceptual loss function utilizing a self-supervised learning algorithm;
(b) generating a loss function comprising the perceptual loss function in (a) and a second loss function; and
(c) optimizing parameters of the deep learning model using the loss function, wherein the deep learning model is trained to enhance a quality of a medical image.

2. The computer-implemented method of claim 1, wherein the self-supervised learning algorithm comprises a contrastive learning.

3. The computer-implemented method of claim 2, wherein the contrastive learning comprises generating a set of negative pairs and a set of positive pairs as training datasets for training the backbone model.

4. The computer-implemented method of claim 3, wherein the set of positive pairs comprise a set of contiguous slices from a same sample.

5. The computer-implemented method of claim 4, further comprising assigning different weights to the set of positive pairs based at least in part on a similarity between the set of contiguous slices.

6. The computer-implemented method of claim 4, wherein the similarity between the set of contiguous slices is based at least in part on a separation of the slices.

7. The computer-implemented method of claim 3, wherein the set of negative pairs comprise a set of contiguous slices from different samples.

8. The computer-implemented method of claim 7, further comprising assigning different weights to the set of contiguous slices based at least in part on absolute difference of normalized slice indices.

9. The computer-implemented method of claim 1, wherein the medical image comprises a Positron Emission Tomography (PET) image or a computed tomography (CT) image acquired at a lower dose.

10. The computer-implemented method of claim 1, wherein the deep learning model is trained to predict an output medical image having a quality that is higher than a quality of the medical image as an input to the deep learning model.

11. The computer-implemented method of claim 1, wherein the second loss function is an organ-specific loss.

12. A computer-implemented method for training a model for perceptual loss, the method comprising:
(a) generating a set of negative pairs and a set of positive pairs as training datasets, wherein the set of positive pairs comprise a set of contiguous slices from a same sample, and wherein the set of negative pairs comprise a set of contiguous slices from different samples; and
(b) training, using a contrastive learning algorithm, a backbone model for the perceptual loss based on the training datasets by tuning parameters of the backbone model, wherein the perceptive loss is used to train a deep learning model for improving a quality of an image data.

13. The computer-implemented method of claim 12, wherein the set of positive pairs are generated by applying a transformation to a volume image of the same sample.

14. The computer-implemented method of claim 12, wherein the contrastive learning algorithm comprise computing a similarity matrix for the set of positive pairs and the set of negative pairs.

15. The computer-implemented method of claim 12, wherein the contrastive learning algorithm comprises assigning different weights to the set of positive pairs based at least in part on a similarity between the set of contiguous slices.

16. The computer-implemented method of claim 15, wherein the similarity between the set of contiguous slices is based at least in part on a separation of the slices.

17. The computer-implemented method of claim 12, wherein the contrastive learning algorithm comprises assigning different weights to the set of contiguous slices based at least in part on absolute difference of normalized slice indices.

18. The computer-implemented method of claim 12, wherein the image data comprises a Positron Emission Tomography (PET) image or a computed tomography (CT) image acquired at a lower dose.

19. The computer-implemented method of claim 12, wherein the deep learning model is trained to predict an output image having a quality that is higher than a quality of the image data as an input to the deep learning model.

20. The computer-implemented method of claim 12, wherein the backbone model is a convolutional neural network.
